(19) United States
(12) Patent Application Publication
(10) Pub. No.: US 2022/0062506 A1
(43) Pub. Date: Mar. 3, 2022

(54) System and Method for Seeding an Autologous Tissue Graft

(71) Applicant: PSIRE MEDICAL, LLC, Arlington, TX (US)

(72) Inventors: Marcus Baker, Olive Branch, MS (US); Ryan Christopher Dregalla, Johnston, CO (US)

(21) Appl. No.: 17/412,849

(22) Filed: Aug. 26, 2021

Related U.S. Application Data
(60) Provisional application No. 63/070,613, filed on Aug. 26, 2020.

(51) Int. Cl. A61L 27/38; A61L 27/24; A61L 27/36; A61K 35/33

(52) U.S. Cl. CPC A61L 27/38; A61L 27/24; A61K 35/33; A61L 27/3687; A61L 27/3691; A61L 27/3604

(57) ABSTRACT

A skin patch having dermal, epidermal, or adipose cell fibroblasts seeded on both the stromal and epithelial side of a scaffold. The scaffold may be an amniotic or collagen membrane or the like. A skin or adipose sample is collected from the patient and enzymatically treated and cultured providing dermal or adipose fibroblasts. The fibroblasts are seeded on the first side of the scaffold and after about 3 hours the second side of the scaffold is seeded. The seeded scaffold may be applied to a patient's wound after about 24-48 hours. This method allows a small one-centimeter by one-centimeter sample of a patient's own skin to grow a patch of skin that is one hundred times larger than the initial sample within as few as three days. This skin graft is made up of cells that were originally from the same patient and creates skin tissues that are genetically identical to the patient.

### CROSS-REFERENCE TO RELATED APPLICATIONS

This application claims benefit from currently pending U.S. Provisional Application No. 63/070,613 titled “System and Method for Seeding an Autologous Tissue Graft” and having a filing date of Aug. 26, 2020, all of which is incorporated by reference herein.

### BACKGROUND OF THE INVENTION

### Field of the Invention

The invention relates generally to methods for culturing cells for tissue models, specifically to skin dressings or patches for coating, healing, cicatrizing, or lessening cutaneous loss in patients.

### Description of Related Art

Currently deep burns, as well as other cutaneous tissue losses due to different traumas or diseases, cause skin injuries which require care coating these areas. Such coating has been carried out with skin grafts directly obtained from the patient's own skin. The process, although effective, generates an important scar in the area where the graft is obtained (thighs, back, buttocks) (photo 1). Furthermore, this process must be carried out under surgery with general or partial anesthesia which generates inherent complications to any as Surgery procedure Such as: severe pain, infection, and bleeding, new surgeries and, although in a very low percentage, death.

Healing large areas of damaged skin can be facilitated with skin patches. Regenerative skin patches are typically comprised of keratinocytes on a fibroblast feeding layer. Some success has been had providing skin patches provided by donors or from cadavers. These skin patches have the same disadvantages of other donated tissue—the body identifies the new skin as foreign and an immune response can prevent the new skin from facilitating healing. Keratinocyte cultures have also been developed using the patient's own serum (autologous), on fibroblasts that have also been harvested from the patient (autologous) as the feeding layer. Since these patches require the patient's own tissues as donor cells, preparation of autologous skin patches require significant time to prepare while the patient is suffering from damaged skin.

So as to reduce the complexity and length of the Detailed Specification, and to fully establish the state of the art in certain areas of technology, Applicant(s) herein expressly incorporate(s) by reference all of the following materials identified in each numbered paragraph below. The incorporated materials are not necessarily “prior art” and Applicant(s) expressly reserve(s) the right to swear behind any of the incorporated materials.
Zamrano Burgl et al., U.S. Pat. No. 9,222,076, Process for the Production of Patches or Dressings of Autologous Skin Through Cultivation of Autologous Keratinocytes and Fibroblasts with Autologous Serum for the Generation of Skin.
Marquette et al., U.S. Pat. No. 9,243,223, High-Density Spot Seeding For Tissue Model Formation.

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), applicant(s) will amend the specification to expressly recite the essential material that is incorporated by reference as allowed by the applicable rules.

### BRIEF SUMMARY OF THE INVENTION

The present invention provides among other things, a skin patch having dermal, epidermal, adipose, or bone marrow-derived cell fibroblasts seeded on both the stromal and epithelial side of a scaffold. The scaffold may be human-derived (allogenic (e.g. amniotic membrane) or autologous), xenograft collagen-based membrane, synthetic or the like.

This method allows a small one-centimeter by one-centimeter sample of a patient's own skin to grow a patch of skin that is one hundred times larger than the initial sample within as few as three days. This skin graft is made up of cells that were originally from the same patient and creates skin tissues that are genetically identical to the patient. This allows for all the benefits of autografting without creating a large donor site that may not heal well.

is an object of the invention to provide a skin patch that does not require a large harvested sample from a patient.

It is an object of the invention to provide a skin patch with higher tensile strength than common skin patches.

is another object of the invention to provide a skin patch made substantially of the patient's own cells.

It is another object of the invention to provide an autologous skin patch more quickly than existing skin patches.

It is another object of the invention to provide a skin patch that may be secured to the patient using an overlaying bandage, sutures, staples, or glue.

It is another object of the invention to provide a skin patch seeded with autologous cells derived from a biopsy of only one square centimeter.

It is another object of the invention to provide a skin patch greater than 2 cm², and preferably greater than 50 cm² in size.

It is another object of the invention to provide a skin patch that is seeded with autologous cells derived from an adipose tissue sample.

It is another object of the invention to provide a skin patch that is seeded with autologous cells derived from bone marrow.

The above and other objects may be achieved using devices involving a patch to improve healing of a wound on a patient, including a scaffold having a stromal side and an epithelial side, a culture prepared from a biopsy of the patient seeded onto the stromal and epithelial side of the membrane. The membrane may be human-derived (allogenic (e.g. amniotic membrane) or autologous), xenograft collagen-based membrane, synthetic or the like summarized in Table 1 below.

TABLE 1

| | | Tissue | |
|---|---|---|---|
| Name | Manufacturer | Source | Material |
| Alloderm | LifeCell | Human | Acellular dermis |
| AlloMax | Bard | Human | Acellular dermis |
| Flex HD | Ethicon/ | Human | Acellular dermis |
| | Musculoskeletal | | |
| | Transplant | | |
| | Foundation | | |
| DermaMatrix | Musculoskeletal | Human | Acellular dermis |
| | Transplant | | |
| | Foundation | | |
| Permacol | Covidien | Porcine | Acellular dermis |
| CollaMend | Davol/Bard | Porcine | Acellular dermis |
| Strattice | KCI/LifeCell | Porcine | Acellular dermis |
| XenMatrix | Brennan Medical | Porcine | Acellular dermis |
| Surgisis | Cook | Porcine | Small intestine sumucosa |
| Surgisis Gold | Cook | Porcine | Small intestine sumucosa |
| Lyosis | Cook | Porcine | Lyophilized small intestine |
| | | | submucosa |
| FortaGen | Organogenesis | Porcine | Small instestine submucosa |
| SurgiMend | TEI Bioscience | Bovine | Fetal dermis |
| Periguard | Synovis | Bovine | Pericardium |
| Veritas | Synovis | Bovine | Pericardium |
| Tutomesh | Tutogen | Bovine | Pericardium |
| Tutopatch | Tutogen | Bovine | Pericardium |

The culture may be fibroblasts that are cultured from the patient's dermal, bone marrow or adipose tissue. The patch may be secured to the patient using at least one of an overlaying bandage, a suture, a staple, or glue.

above and other objects may be achieved using methods involving taking a sample of skin or adipose tissue from the patient, enzymatically, mechanically, or ultrasonically treating the sample, and growing a culture from the treated sample. The culture is then used to seed first one side, and then the other side of a scaffold. The scaffold may be flipped, and the second side seeded after at least 1 hour, and the scaffold can be applied to a wound of a patient after at least 16 hours. In a preferred embodiment, the scaffold is flipped between 3 and 4 hours after the first side of the scaffold is seeded and the scaffold is applied to the wound between 24 and 48 hours after the sides of the scaffold are seeded. The seeding may be high density and/or in combination with low density seeding.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts the dermis primary culture from sample 1 at day 7 at a 2× objective.

FIG. 2 depicts the dermis primary culture from sample 1 at day 7 at a 10× objective.

FIG. 3 depicts a flow cytometry analysis of the Isotype Control (Negative) of dermal fibroblasts labeled for CD90 in Sample 1.

FIG. 4 depicts a flow cytometry analysis of the dermal fibroblasts labeled for CD90 in Sample 1.

FIG. 5 depicts the Dermal Cell Foci and Low Density Seeding onto Membrane of Sample 1 on day 4 at 2× objective.

FIG. 6 depicts the Dermal Cell Foci and Low Density Seeding onto Membrane of Sample 1 on day 4 at 4× objective.

FIG. 7 depicts a fluorescent image of contracting foci of Sample 1.

FIG. 8 depicts the Dermal Cell Foci and Low Density Seeding onto Membrane of Sample 1 on day 7 at 4× objective.

FIG. 9 depicts the dermis primary culture of sample 2 on day 6.

FIG. 10 depicts a flow cytometry analysis of the Isotype Control (Negative) of dermal fibroblasts labeled for CD90 in Sample 2.

FIG. 11 depicts a flow cytometry analysis of the dermal fibroblasts labeled for CD90 in Sample 2.

FIG. 12 depicts the dermal cell foci seeded at 15,000 k cells on membrane stromal side on day 3 for Sample 2.

FIG. 13 depicts the dermal cell foci seeded at 2,000 cells per cm2 on membrane stromal side on day 3 for Sample 2.

FIG. 14 depicts the dermal cell foci seeded at 15,000 k cells on membrane stromal side on day 6 for Sample 2.

FIG. 15 depicts the dermal cell foci seeded at 2,000 cells per cm2 on membrane stromal side on day 6 for Sample 2.

FIG. 16 depicts the dermal cell foci seeded at 2,000 cells per cm2 on membrane epithelial side on day 2 for Sample 2.

FIG. 17 depicts the epidermis primary culture at the center of the flask for sample 2 on day 13.

FIG. 18 depicts a flow chart of the process of culturing and applying the patch of a particular embodiment of the invention to a patient.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

The present invention relates to a system and process for forming skin dressings or patches for coating, healing, cicatrizing, lessen cutaneous loss in patients. Said process is described below based on an exemplary embodiment provided to explain the invention as to understand the same in all its scope and spirit. Referring to FIG. 18, in one embodiment the process is based in obtaining a skin sample and an adipose sample of the patient and with these two elements the skin is cultured, which is placed on an amniotic membrane, collagen patch, or the like, thus producing a dressing which further on will be used on a patient when required. In one embodiment, a kit is supplied to a physician to take a skin sample and/or an adipose sample from a patient who has had an injury to the skin. The biopsy size of the sample acquired can be approximately lcm. A sample of adipose tissue is advantageous because it is plentiful in most patients and is easily acquired from the patient. The skin and adipose sample is sent to a lab, where it is enzymatically treated, mechanically or ultrasonically dissociated to prepare sample tissue.

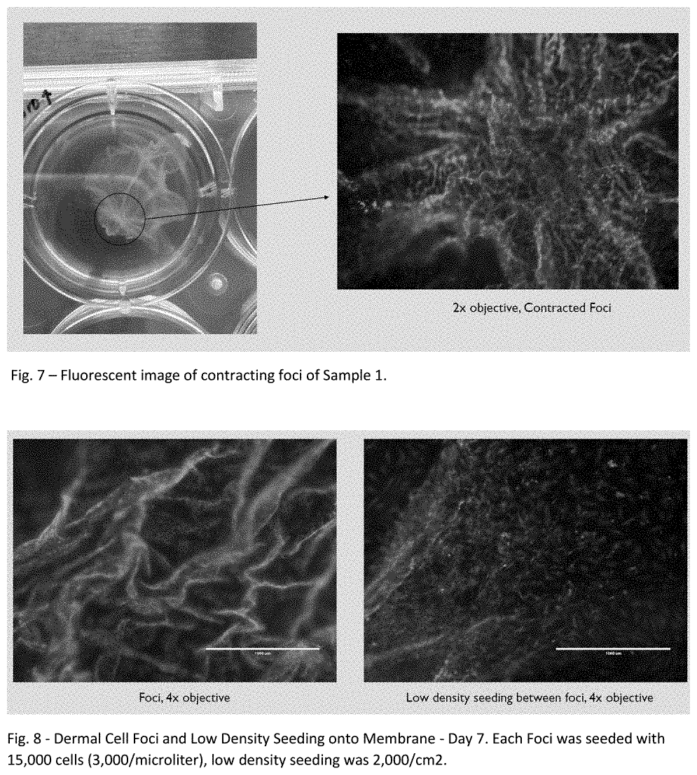

A scaffold such as an amniotic membrane, collagen patch, or the like “scaffold” is prepared for seeding and seeded with cells grown from a sample obtained from the patient. The cells may be dermal fibroblasts/stromal/stem cells, epidermal fibroblasts, keratinocytes, adipose-derived fibroblasts/stem cells, bone marrow stromal/stem cells/fibroblasts. The cells may be uniformly seeded at a “low-density” or may be seeded using a “high-density” seeding technique to create foci. The seeding of both sides of the scaffold is done to produce an orientation specific, chiral biologically active scaffold suitable for transplantation. Initially, cells are seed at a “low density” on the epithelial or, “upward facing” side of the scaffold; the cell censity can range from 1,500-6,500 cell per cm² of scaffold while the scaffold is in total cell culture media. After between 1.5 hours and 48 hours, the scaffold is flipped. The now stromal side, which will be oriented to face the wound bed is upward. Media is removed, leaving enough in the culture vessel to retain hydration without flowing over the scaffold. Stromal cells/fibroblasts are then seeded using the high-density seeding method to create foci. This is achieved by preparing a liquid suspension of the cells ranging from a cell density of 3,000-30,000 cells per microliter (μl). From this mixture, 2 μl-8 μl of the cell suspension is seeded every 5 cm² of scaffold. Cells are incubated for 1.5 hours-24 hours under these conditions to allow adhesion. In thin membranes (<100 microns), such as amniotic membrane, the foci are macroscopically evident by forming a “pinched” region in the tissue, indicating successful cell adhesion (FIG. 7). Finally, additional media is added to the culture vessel and the stromal side is further seeded at a low density as previously described. The product is now prepared to be shipped.

In the high-density seeding technique, cells are seeded at a selected concentration on a support to form a cell spot, and the cells are incubated to allow the cells to partially attach. The cells are then rinsed to remove any cells that have not partially attached, and culture medium is added to enable the cells to proliferate at a periphery of the cell spot and to differentiate toward the center of the cell spot. See U.S. Pat. No. 9,243,223 to Marquette et al. The low-density seeding allows the active proliferation of the cells across the scaffold.

When the patch is received by the patient's physician, the patient's wound may then be debrided and prepared and the patch applied to the wound. A silicone membrane is applied over the patch and wound area and a traditional bandage applied to hold the patch in place. The patch may also be applied to the wound using a suture, staple, glue, or any other mechanism known by those having skill in the art. The tensile strength of a patch prepared in this manner can be approximately 3-5 times that of an autograft.

The patch and silicone bandage remain in place over the wound for an amount of time to allow the patch to be absorbed by the body to aid in healing the wound, typically about 21 days. During that time, the traditional bandage can be changed as often as preferred by the physician, typically about 2-3 times per week. After that time, epithelialization is complete, and the silicone bandage may be removed. The physician and/or the patient can continue to dress and monitor healing until the wound is completely healed.

The details of the process of preparing the patch of the invention will be described using the following examples:

After acquiring a skin sample, PSIRE dermal and epidermal tissue dissociation and plating was accomplished using the following procedure:

1. Aspirate off shipping Media.

2. Rinse with PBS 2×(—CaCl₂)/—MgCl₂) (Thermo 10010-023).

3. Incubate sample in growth supplement ≥10%, comprised of xeno- (e.g. fetal bovine serum), allo-, or autologously derived serum, plasma, platelet lysate or platelet releasatein αMEM (12571-063) or DMEM with 100 U/mL penicillin-100 ug/mL streptomycin for 30 mins at 37° C.

4. Rinse with PBS 2×.

5. Add 5 mL of 0.2% Dispase to a petri dish with skin sample.

6. Incubate at 37° C. for 1 hour.

7. Pinch or peel sample with forceps separate epidermis from the dermis.
a. Epidermis is grey.
b. Dermis is white.

8. Place dermis into a labeled 15 mL conical tube with 5 mL PBS.

9. Scrape each side of the epidermis with a scalpel blade to disrupt the dead keratinocyte layer and residual basement membrane.

10. Collect the epidermis and fragments/scrapings via serological pipette and 5 mL PBS.

11. Place epidermis and fragments in a labeled 15 mL conical tube.

12. Centrifuge tubes at 500 RCF for 1 min and aspirate off PBS.

13. Add 5 mL PBS to each tube.

14. Repeat centrifugation at 500 RCF for 1 min and aspirate off PBS.

15. Add 1 mL of TrypLE Express (cat #: 12605010) to each tube.

16. Incubate at 37° C. for 30 mins on a shaker.

17. Neutralize with 20% FBS in αMEM at a 1:1 ratio.

18. Remove tissue (dermis and epidermis) from tubes and place in fresh conical tubes.

19. Centrifuge the respective cell suspensions from the TrypLE at 500 RCF for 5 minutes.

20. Aspirate off supernatant.

21. Resuspend pellet in 500 ul 20% FBS in αMEM; obtain total nucleated cell count with DAPI in 0.1% Triton X-100 (TNC).

22. Plate each cell suspension in a separate T-25 flask (25 cm² surface area).
a. Flask labeled “Epidermis”.
b. Flask labeled “Dermis”
   With remaining epidermis tissue:

23. Repeat TrypLE treatment for 15 minutes at 37° C.

24. Repeat neutralization and centrifugation steps and obtain TNC.

25. Add the cell suspension to the flask labeled “Epidermis”.

With remaining dermal tissue, complete the following steps:

26. Digest in 1 mL collagenase solution (collagenase I 0.1%+collagenase II 0.1% in Hank's Balanced Saline Solution)
a. Included epidermis in this step in the initial sample with minimal cell recovery.

27. Incubate at 37° C. on a shaker for 45 minutes.

28. Volume up with 3× the respective volume using PBS.

29. Centrifuge at 500 RCF for 5 mins; aspirate off supernatant.

30. Resuspend pellet in 500 ul 20% FBS in αMEM.

31. Acquire TNC.

32. Add the cell suspension to the “Dermal” T-25 flask.

33. Add TrypLE express to remaining dermal tissue for 15 minutes at 37° C.

34. Neutralize with 20% FBS in αMEM at a 1:1 ratio.

35. Centrifuge at 500 RCF for 5 minutes; aspirate off supernatant.

36. Resuspend pellet in media and obtain a TNC.

37. Plate pellet in “Dermis” T-25 with 20% FBS in αMEM.

38. For residual intact epidermal tissue, plate in a 10 cm diameter cell culture dish labeled “Epidermal explant culture”.

39. For residual intact epidermal tissue, plate in a 10 cm diameter cell culture dish labeled “Dermal explant culture”.

40. Assess cultures over the next 4-7 Days.

Dermal and epidermal explant culture were seeded on amniotic membranes. It was found that epidermal cells did not result in robust, timely growth.

Epidermal Cells Culture

Epidermal cells/keratinocytes were cultured for 13 days in 20% fetal bovine serum/alpha-MEM. Both enzyme treatment and explant cultures did not result in robust, timely growth. Hyperdense colonies emerged in the center of the flask, but throughout most of the flask, cells were sparse, with LESS than 10% of the flask surface area contained adherent cells at Day 13, far beyond our aim of completing culture in less than five days. In addition, cells appear to be fibroblasts-like as opposed to epithelial cells.

The flow cytometry marker chosen, CD90, is a fibroblast and keratinocyte progenitor cell Marker (See, https://www.ncbi.nlm.nih.gov/pubmed/16704635). Calcium is a key modulator of differentiation into keratinocytes and may be added to topically at relevant time point post-transplant (https://www.ncbi.nlm.nih.gov/pmc/articles/PMC3491811/). If adipose-derived human mesenchymal stem cells (ad-MSC) are used in the future, they can promote the proliferation of dermal fibroblast in the wound as well as keratinocytes. Ad-MSCs can differentiate into dermal fibroblasts and keratinocytes (See, https://stemcellres.biomedcentral.com/articles/10.1186/s13287-018-1044-5; https://www.ncbi.nlm.nih.gov/pmc/articles/PMC3846628/)

Total Nucleated Cell Count at each Step Digestion

| | 1st Trypsin | Col 1 & 2 digest | 2nd Trypsin |
|---|---|---|---|
| Sample 1 Epidermis | 155,000 | 16,250 | 200,000 |
| Sample 2 Epidermis | 75,000 | NA | 55,000 |
| Sample 1 Dermis | 43,750 | 80,000 | 103,750 |
| Sample 2 Dermis | 17,500 | 65,000 | 140,000 |

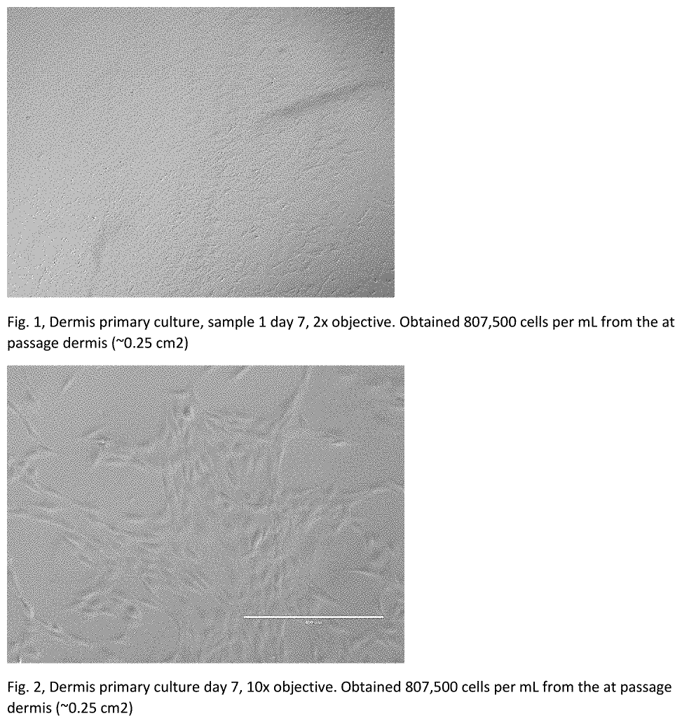

Dermal fibroblasts were found to provide robust, timely growth. Results are shown in FIGS. 1-2.

Seeding Dermal Cell Foci on Stromal Side of Amniotic Membrane Sample 1:

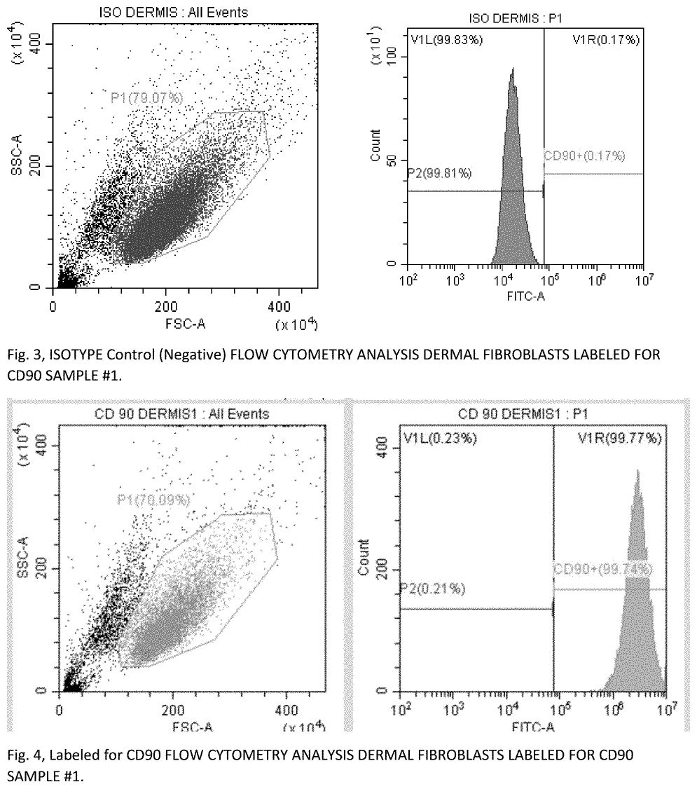

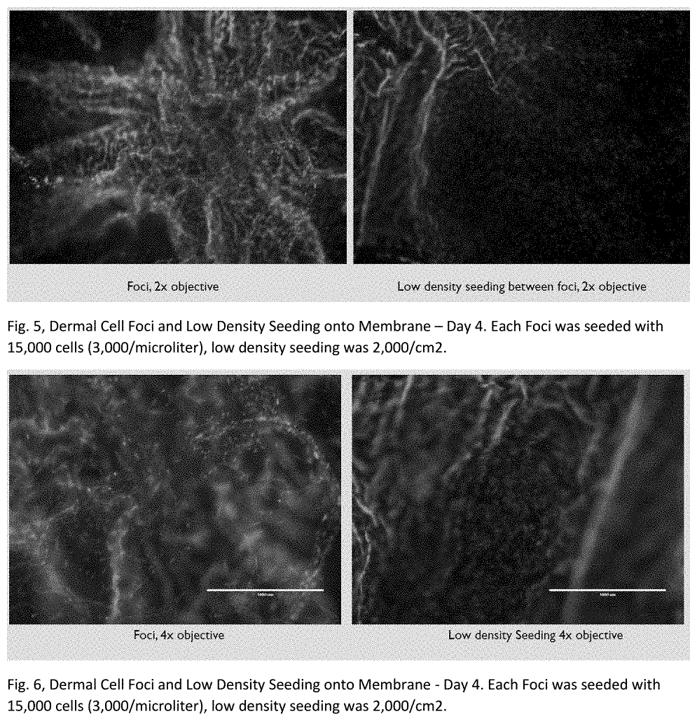

Dermal fibroblasts were cultured for 7 days to achieve confluence 20% fetal bovine serum/alpha MEM. Cells were removed from the flask via trypsin treatment and analyzed via flow cytometry for the fibroblast marker CD90. Amniotic membrane was decellularized; verified by DAPI staining. Human dermal fibroblasts from culture were resuspended at 3,000 cells per microliter (see foci seeding and validation in plates). On the stromal side of the membrane, 15,000 dermal fibroblasts were seeded per 5 cm2 in a 5 ul droplet performed on a 4 cm×4 cm membrane, resulting in 3 foci. Additional dermal fibroblasts were seeded across the membrane at a density of 2,000/cm2 (“low density”). At Days 4 and 7, cells were imaged by fluorescent microscopy via calcein (green=live) and DAPI (blue=dead). Results are shown in FIGS. 3-8.

Dermal Cells Seeding on Stromal Side of Amniotic Membrane. Sample 2:

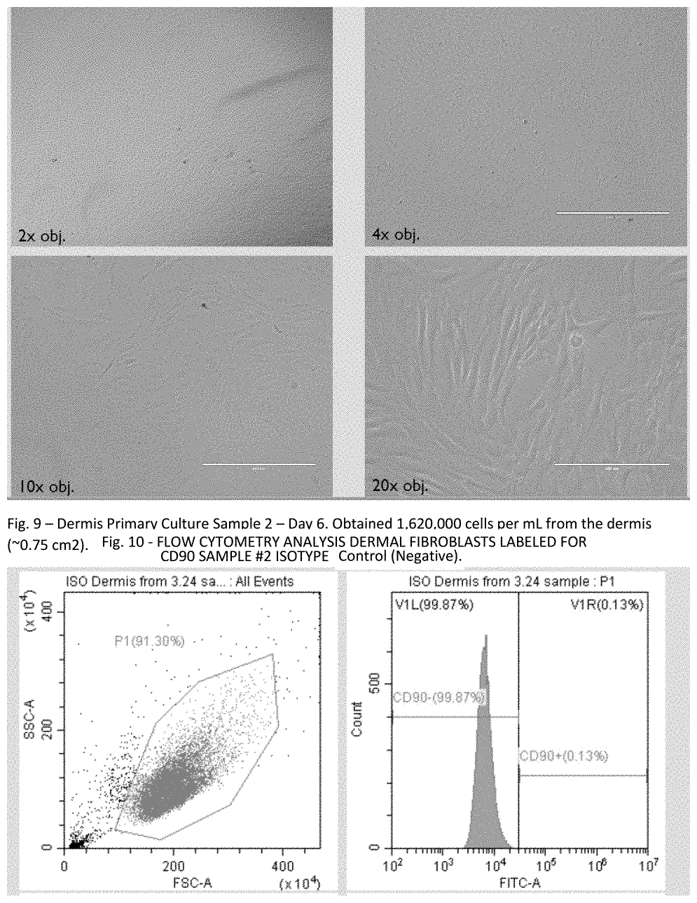

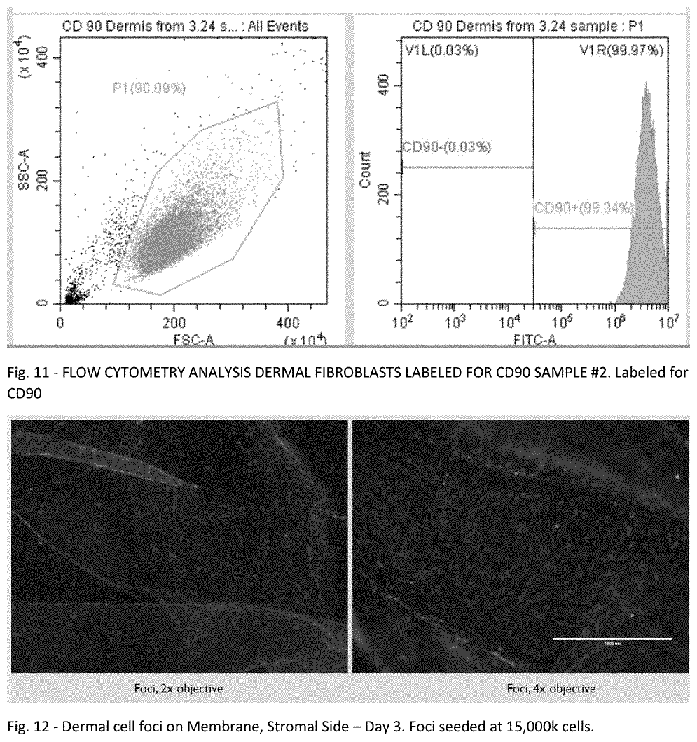

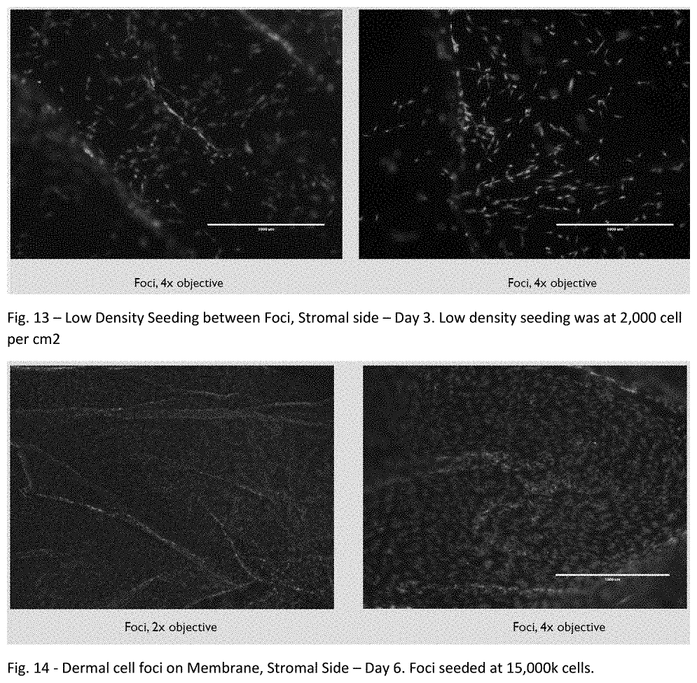

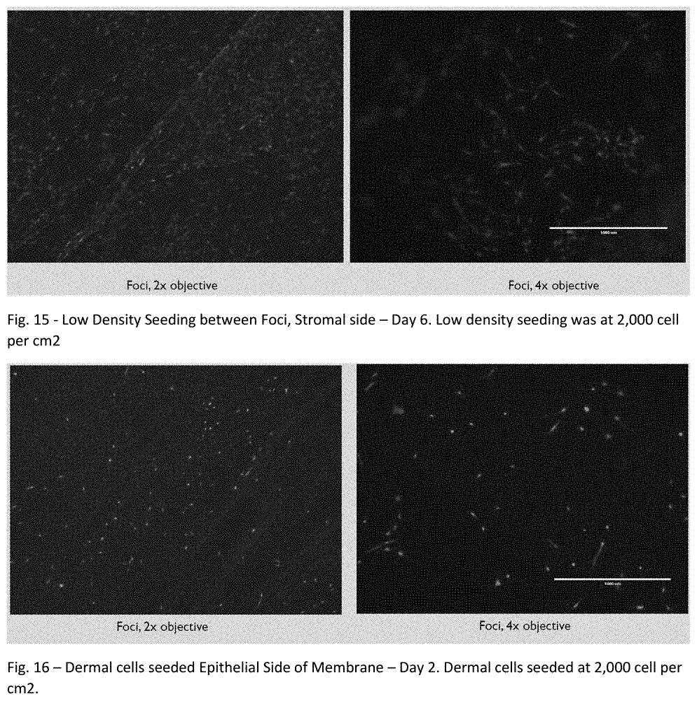

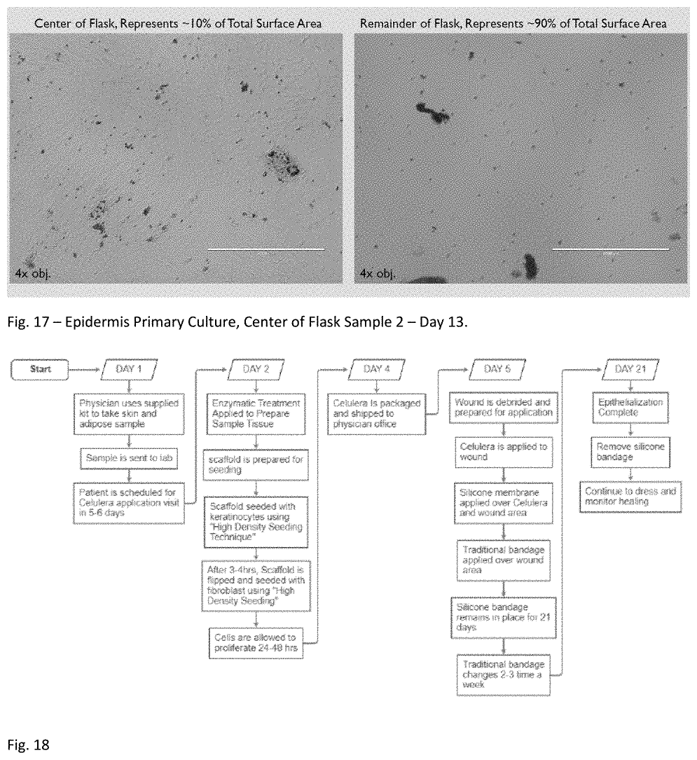

Dermal fibroblasts were cultured for 6 days to achieve confluence in 20% fetal bovine serum/alpha-MEM. Cells were removed from the flask via trypsin treatment and analyzed via flow cytometry for the fibroblast marker CD90. Amniotic membrane was decellularized; verified by DAPI staining. Results are shown in FIGS. 9-15

Cell Attachment on Epithelial Side of the Membrane.

Human dermal fibroblasts from culture were resuspended at 3,000 cells per microliter (see foci seeding and validation in plates). On the stromal side of the membrane, 15,000 dermal fibroblasts were seeded per 5 cm2 in a 5 ul droplet, performed on a 4 cm×4 cm membrane, resulting in 3 foci. Additional dermal fibroblasts were seeded across the membrane at a density of 2,000/cm2 (“low density”). At Days 3 and 6, cells were imaged by fluorescent microscopy via calcein (green=live) and DAPI (blue=dead).

Cell Attachment on Epithelial Side of the Membrane.

Dermal fibroblasts were seeded on the epithelial side at a low density, 2,000 cells per cm2. Results are shown in FIG. 16.

We claim:

1. A patch to improve healing of a wound on a patient, the patch comprising:
a scaffold having a stromal side and an epithelial side;
a culture prepared from a biopsy of the patient seeded onto the stromal and epithelial side of the membrane.

2. The patch of claim 1, wherein the fibroblasts are cultured from the patient's dermal tissue.

3. The patch of claim 1, wherein the fibroblasts are cultured from the patient's adipose tissue.

4. The patch of claim 1 wherein the scaffold is an amniotic membrane.

5. The patch of claim 1 wherein the scaffold is a collagen membrane. Synthetics and xeno?

6. The patch of claim one wherein the scaffold is a synthetic membrane.

7. The patch of claim 1 wherein the patch is coupled to the patient using at least one of an overlaying bandage, a suture, a staple, or glue.

8. A method of treating a wound on a patient, the method comprising:
taking a sample of skin or adipose tissue from the patient;
enzymatically, mechanically, or ultrasonically treating the sample;
growing a culture from the treated sample;
on a scaffold having a first side and a second side, seeding the first side of the scaffold using the culture;
after at least 1.5 hours, flipping the scaffold and seeding the second side of the scaffold;
after at least 16 hours, applying the scaffold to the wound.

9. The method of claim 8, wherein the scaffold is flipped between 1.5-24 hours after the first side of the scaffold is seeded.

10. The method of claim 8, wherein the scaffold is applied to the wound between 24 and 48 hours after the sides of the scaffold are seeded.

11. The method of claim 8, wherein at least one of the sides are high density seeded.
